(19) United States
(12) Patent Application Publication
(10) Pub. No.: US 2004/0265925 A1
(43) Pub. Date: Dec. 30, 2004

(54) Method and kit for the measurement of the activation of basophils induced by allergen to determine hypersensitivity to some substances

(76) Inventor: Marie Havranova, Praha (CZ)

Correspondence Address: MORRISON & FOERSTER LLP, 3811 VALLEY CENTRE DRIVE, SUITE 500, SAN DIEGO, CA, 92130-2332, US

(21) Appl. No.: 10/489,884

(22) PCT Filed: Sep. 10, 2002
(86) PCT No.: PCT/CZ02/00049

(30) Foreign Application Priority Data
Sep. 17, 2001 (CZ) PV 2001-3356

(51) Int. Cl. G01N 33/567

(52) U.S. Cl. 435/7.21

(57) ABSTRACT

A method for the measurement of the activation of the basophils following stimulation with an allergen to determine hypersensitivity to some substances, in which a blood sample with an addition of interleukin-3 in a quantity of 0.05 to 50 ng/ml based on the volume of the sample, and of appropriately diluted allergen in a quantity of 0.5 to 100 units/ml is incubated at the temperature corresponding to the physiological environment for 15 to 45 minutes, whereafter a staining with anti-CD63.antibody in an amount of 3 to 30 μl/100 μl of blood and with the antibody against a receptor of the basophil in an amount of 3 to 30 μl/100 μl of blood are added at a temperature of 0 to +10° C. and the sample, after vortexing, is then incubated in an ice bath for 15 to 30 minutes, and then is lysed and subjected to flow cytometry.

### TECHNICAL FIELD

The invention pertains to a method for determination of the response of the basophils in a patient's blood following stimulation with an allergen in order to determine hypersensitivity to some substances by means of double labelling.

### BACKGROUND ART

In order to detect hypersensitivity of an organism to some substances either in vivo tests (skin tests, dual blind trial) or in vitro tests are being used. Currently, the in vitro tests tend to prevail, so that the allergenic burden to the patient is avoided. Other reasons in favour of the in vitro tests are the patient's age, fear of a hypersensitive reaction, patient's comfort (unlike the skin punctures wherein one stick corresponds to determining one allergen, the in vitro tests make it possible to determine several allergens in one collection of the blood).

At present, the most widely used in vitro test is the measurement of the level of specific IgE antibodies (sIgE) in the patient's blood, i.e. of the end product of the cell's specific response. The most widespread principle of the test is the enzyme-linked immuno-assay (ELISA) in various modifications. The determination can be made with a time delay after patient's blood collection. From a single collection antibodies against more allergens can be determined. However, the results may sometimes differ from the skin tests. For example, when the patient has not come into contact with the allergen for some time, the antibodies may disappear, even when the hypersensitivity pertains. Also, cross-reacting antibodies are often being found and the method can generally be poorly standardized.

This is why recently methods that monitor an immediate response of the cells to stimulation have started to be developed. Currently much attention has been paid to the basophils. They are specialized effector cells of the immune system, playing an important role in the allergic reaction. Sensitized cells are activated by the specific allergen The activation manifests, on one hand, by expression of some receptors on their surface (CD63), and, on the other hand, by production of the cytokines (which support production of sIgE) and mediators (such as histamine, leukotrienes, etc.). They are responsible for some clinical manifestations of allergy.

These methods are still far from being much widespread. Currently, histamine and sulphidoleukotrienes can be determined in the supernatant following stimulation of the cells with an allergen by ELISA tests. A disadvantage is the necessity of the separation of the cells. In course of it, non-specific activation may occur. In addition, the patient must not use any antihistamine drugs for at least 48 hours.

Determination of the expression of the activation antigen CD63 on the basophils has the following advantages: 1) work with whole blood, 2) use of soluble allergens, the same ones as for skin tests, 3) possibility to determine type 1 hypersensitivity to an allergen, even when no sIgEs are present in the peripheral. Moreover, the cells are not activated by the cross-reacting antibodies to allergen because of their low affinity. But detection of the basophils by staining with anti-IgE has some limitations. At a very low level of overall IgE (<80 IU/ml) the basophils can only be stained with much difficulty (low fluorescence) and that at a high level of IgE the antibody (anti-IgE) added can be bound by serum IgE.

Introduction of the flow cytometry, i.e. of the method which enables to label the cells by means of surface receptors, will make it possible to develop methods that are based on monitoring of the cellular response. Thus, this method makes it possible to determine the immediate response of the basophils to the allergen in vitro. In the peripheral blood, the percentage of the basophils is very low (about 1%). They belong to the group of granulocytes, with which they share most surface antigen. Currently, the fact that they—unlike other cells—bear the FcεRI receptors on their surface and bind IgE. That is used for their staining with antibodies anti-IgE. Therefore, the basophils are being labelled with a fluorescent antibody against IgE (cf. the Basotest produced by Orpegen Pharma).

### SUBSTANCE OF THE INVENTION

We believe that the determination of the activation of basophils is a method that approximates the in vivo conditions most, we have attempted to overcome the disadvantages of staining of the basophils by means of anti-IgE antibodies. We have found that it is much more advantageous to label the basophils with an antibody that is against a surface receptor which is specific for them. Thus, disadvantages, associated mainly with the low level of IgE, can be overcome, as this labelling is independent from said level. It is known at present that the receptor for interleukin-3 (IL-3) is expressed on the basophils and bears the designation CD123. For that reason it is possible to select a fluorescence labelled antibody that has been prepared against this receptor in order to stain the basophils. CD203c may be another surface marker for staining the basophils, such that an anti-CD203c antibody can be used.

The determination is carried out in the whole blood. After the allergen is added and during the subsequent incubation the allergen binds itself to the IgE antibodies that are specific against the given allergen in the case of the cells sensitive to the allergen added. The antigen-antibody bond will instruct the cells to start the activation and thus also to start up the processes that end in degranulation of the basophils and release of the mediators, responsible for the allergic reaction in vivo. The response of the basophils is monitored by the measurement of the expression of CD63 antigen. The basophils are stained with an antibody against the receptor that they bear on their surface, preferably with an antibody against the CD123 (receptor for IL-3) or against the CD203c receptor. If there are no antibodies against the added allergen bound on the patient's basophils, no antigen-antibody reaction will arise and thus no activation of the cells will occur, and hence no expression of CD63 will take place.

### BRIEF DESCRIPTION OF THE DRAWINGS

The annexed figures illustrate sets of histograms for each patient tested, obtained by means of the Coulter Epics XL flow cytometer.

The meanings of the individual histograms: Histogram 1—Distribution of the cells according to light scattering in the forward (FS) and side direction (SS). Gate N delineates the area wherein the basophils are present.

Histogram 2—Delineation of the basophil domain based on the light scattering in the side scatter (SS) and on the binding of the anti-IgE/FITC antibody (gate F)

Histogram 3—Delineation of the basophil domain based on the fight scattering in the side direction (SS) and on the binding of the anti-CD123 (or anti-CD203c/PE) antibody (gate A)

Histogram 4—Distribution of the cells by the fluorescence intensity, i.e. the anti-IgE/FITC binding.

Histogram 5—Distribution of the cells by the fluorescence intensity, i.e. the anti-CD123/PE (or anti-CD203c/PE) binding.

Histogram 6—Percentage distribution of the basophils that are stained with anti-IgE/FITC (gate F, Histogram 2)

Quadrant 1 (B1)—The cells stained with the anti-IgElFITC antibody—other cells than basophils present in gate F

Quadrant 2 (B2)—The cells labelled with both the anti-CD123/PE (or anti-CD203c/PE) antibody and the anti-IgE/FITC antibody

Quadrant 3 (B3)—Unstained cells

Quadrant 4 (B4)—The cells stained with the anti-CD123/PE (or anti-CD203c/EP) antibody

Histogram 7—Percentage distribution of the basophils that are anti-CD123/PE stained (gate A, Histogram 3)

Quadrant 1 (G1)—The cells stained with the anti-IgE/FITC antibody—other cells than basophils present in gate F

Quadrant 2 (G2)—The cells stained with both the anti-CD123/PE (or anti-CD203c/PE) antibody and the anti-IgE/FITC antibody

Quadrant 3 (G3)—Unstained cells

Quadrant 4 (G4)—The cells labelled with the anti-CD123/PE (or anti-CD203c/PE) antibody

Histogram 8—Distribution of the basophils that are anti-CD123/PE (or anti-CD203c/PE) labelled based on the light scattering in the side direction (SS) and on the binding of the anti-IgEFITC antibody.

FIGS. 1-1 to 1-8 illustrate the common expression of CD123+/IgE+ on the basophils as a function of the concentration of the overall IgE in the serum, summarized in Table 1.

FIGS. 2-1 to 2-5 illustrate the common expression of CD203c+/IgE+ on the basophils as a function of the concentration of the overall IgE in the serum, summarized in Table 2.

FIGS. 3-1 to 3-4 illustrate:

FIG. 3-1—Sample without stimulation (negative control of K0)

FIG. 3-2—Sample stimulated with FMLP, non-specific control)

FIG. 3-3—Sample stimulated with wasp allergens

FIG. 3-4—Sample stimulated with honey bee allergens

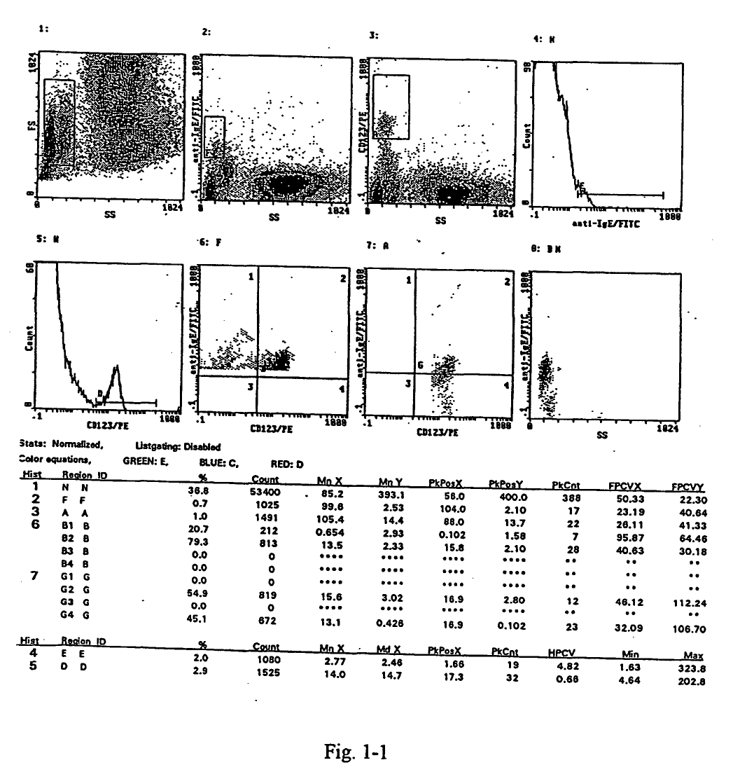

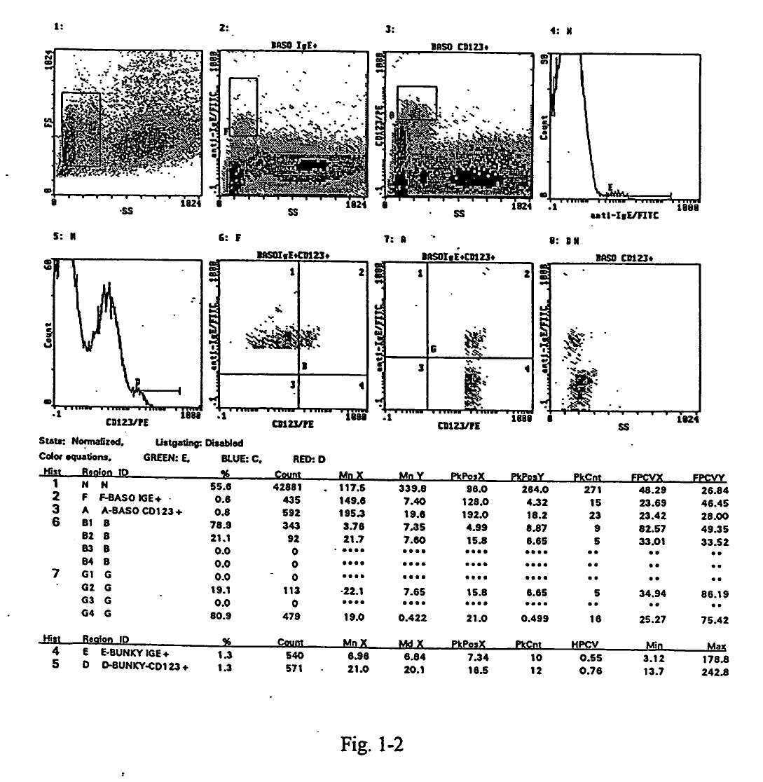

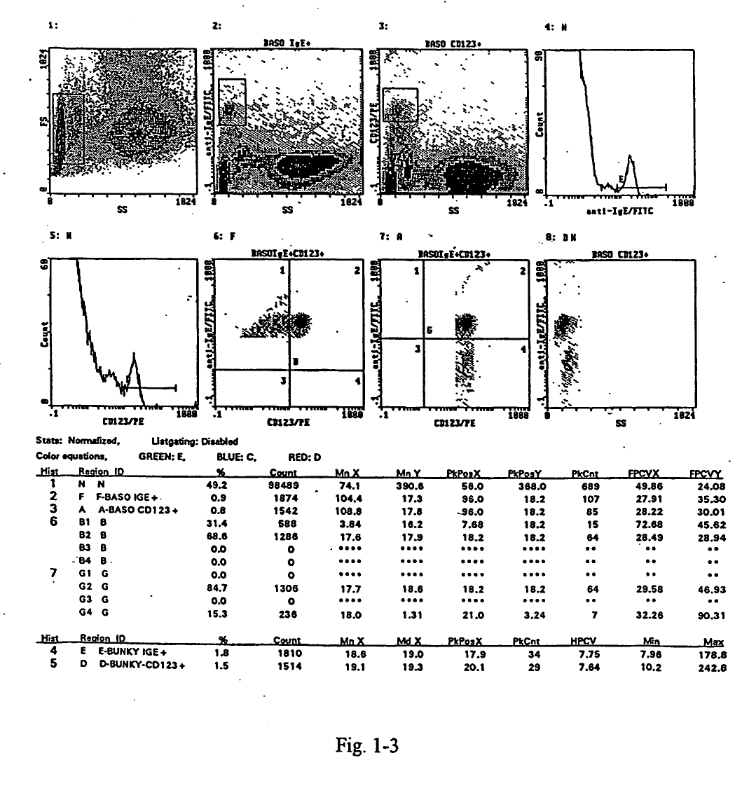

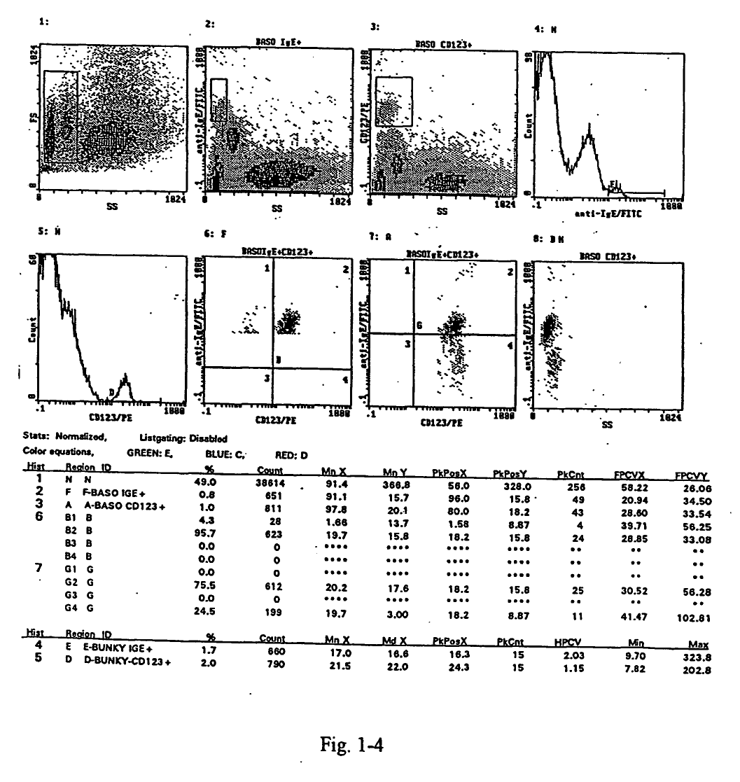

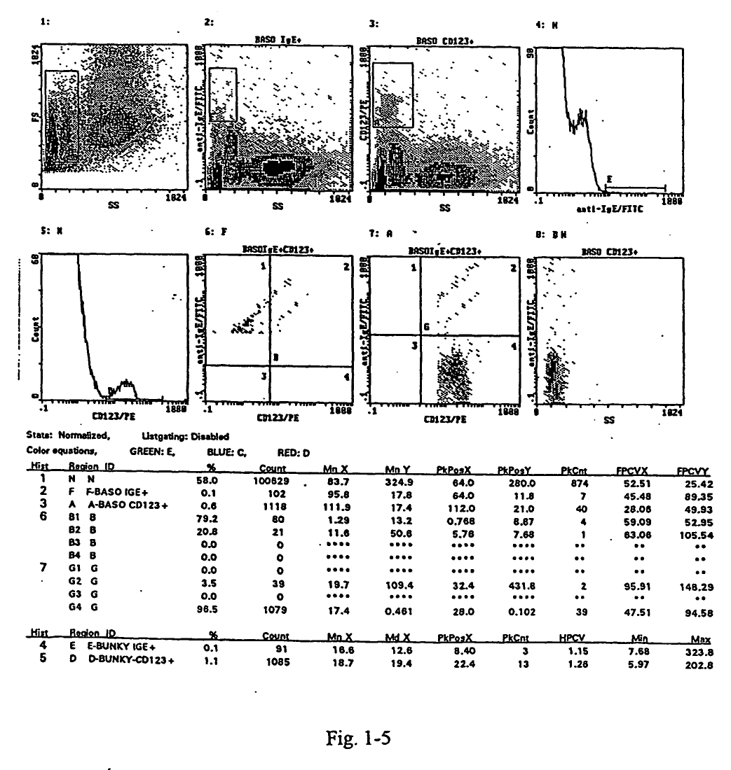

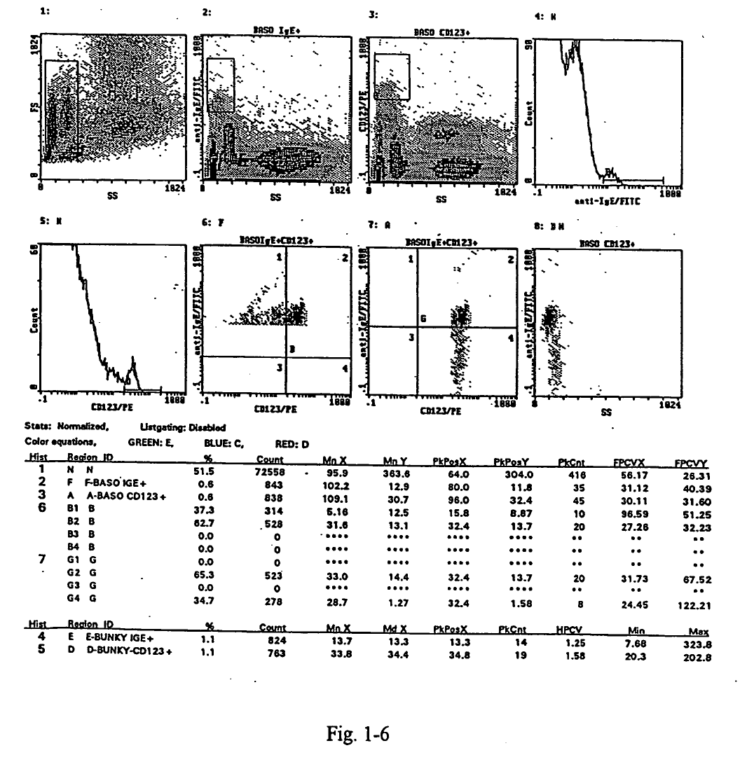

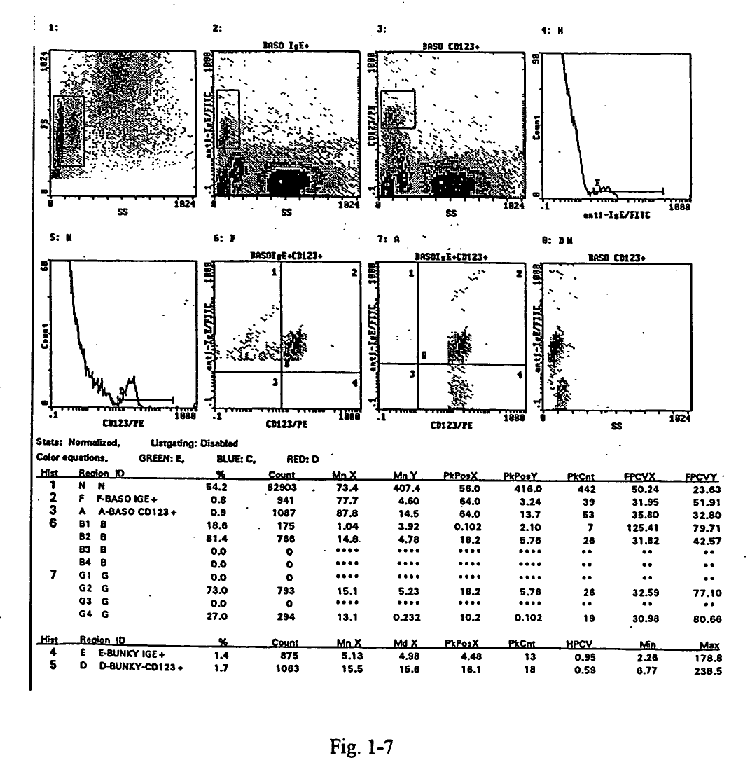

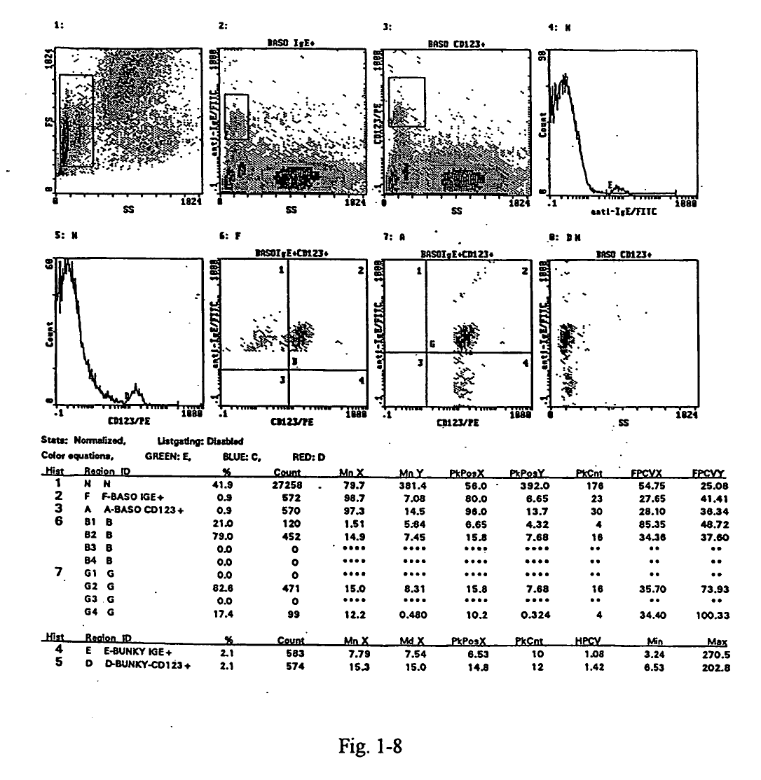

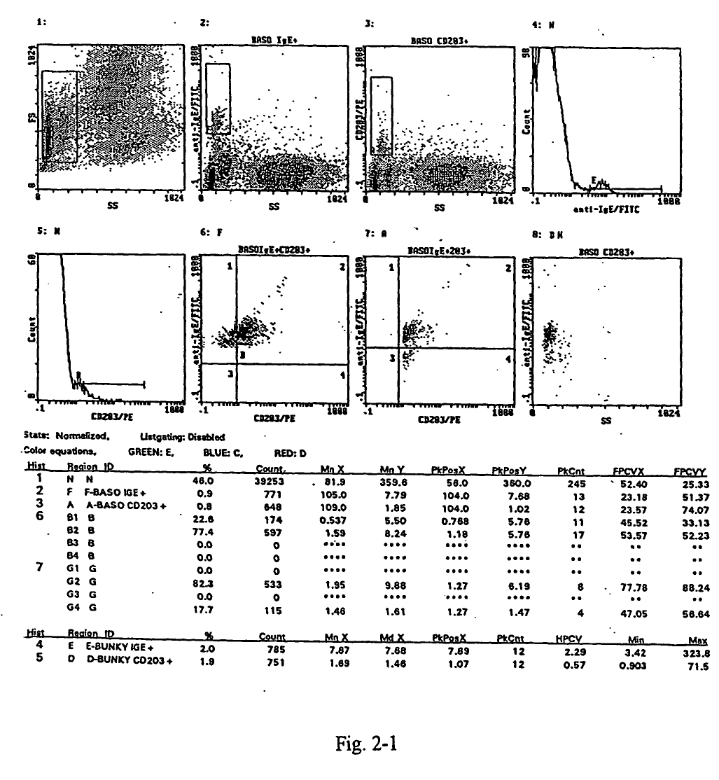

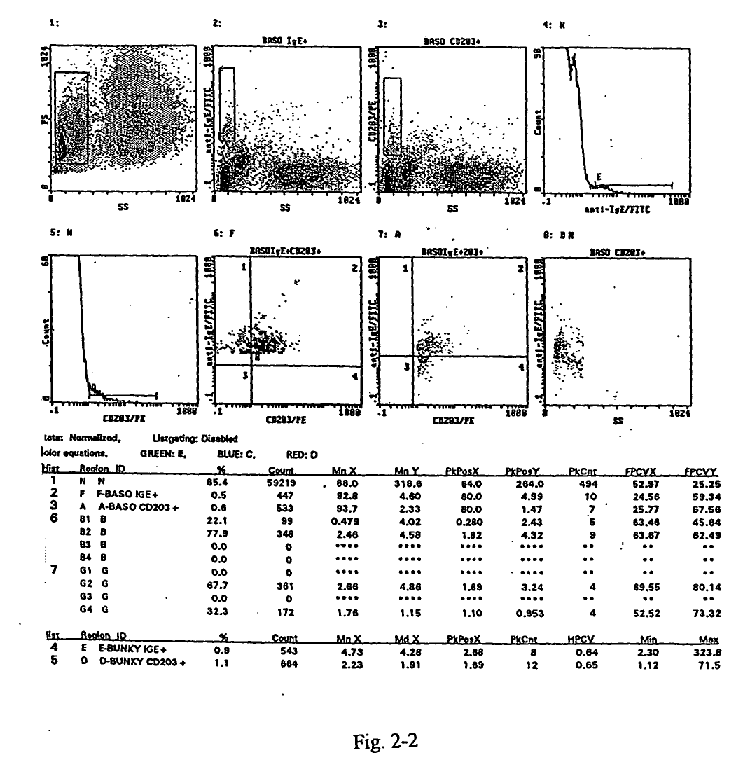

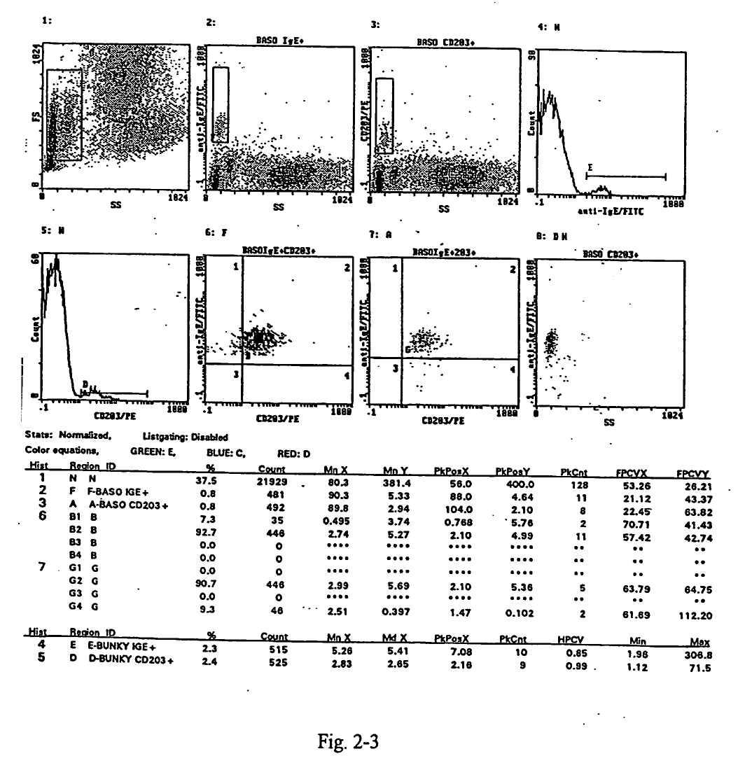

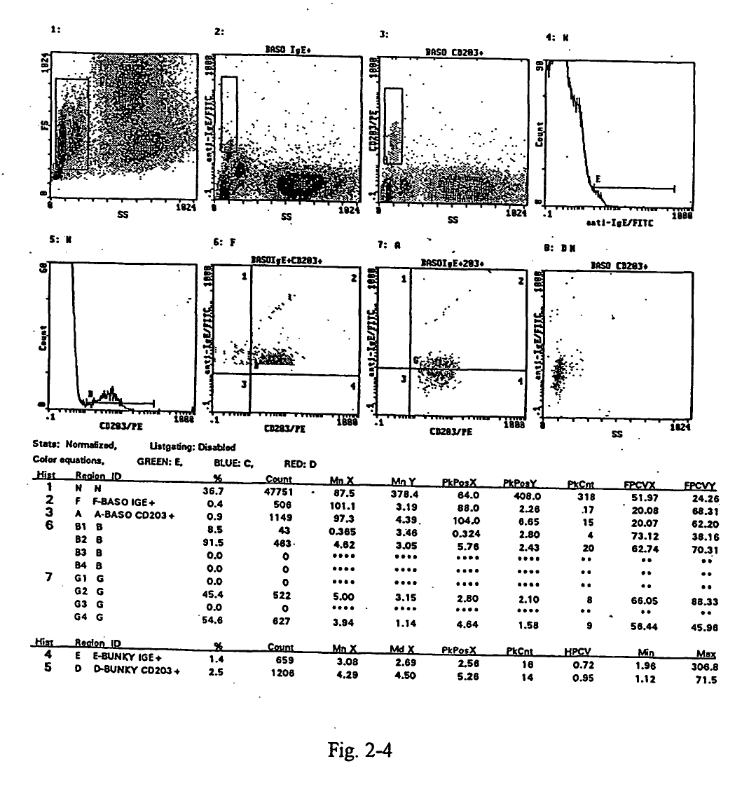

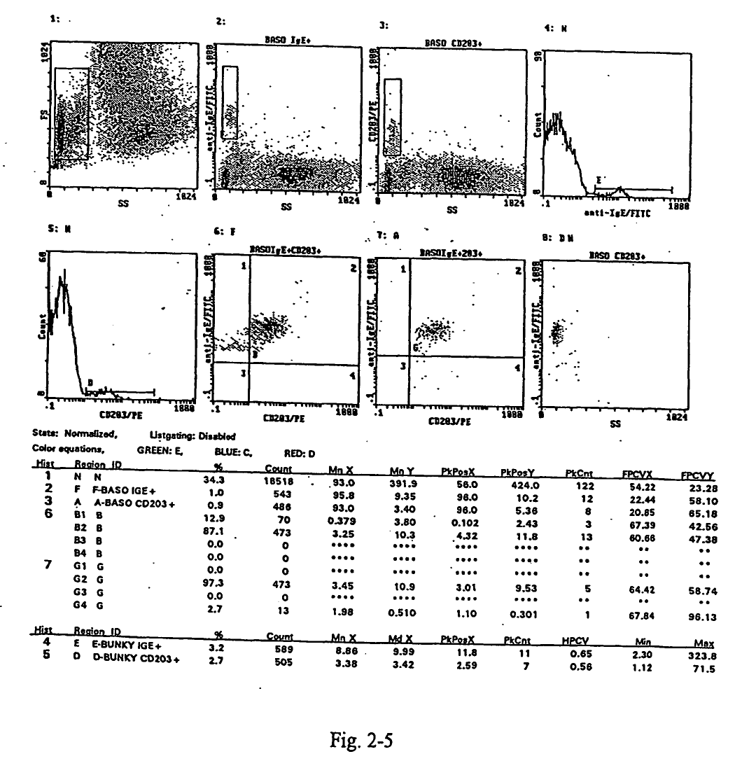

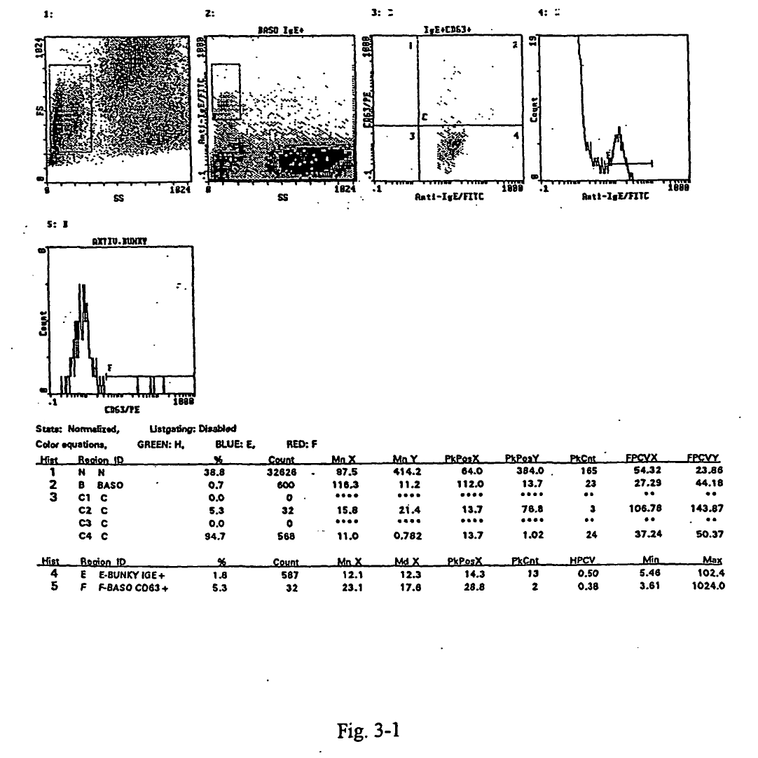

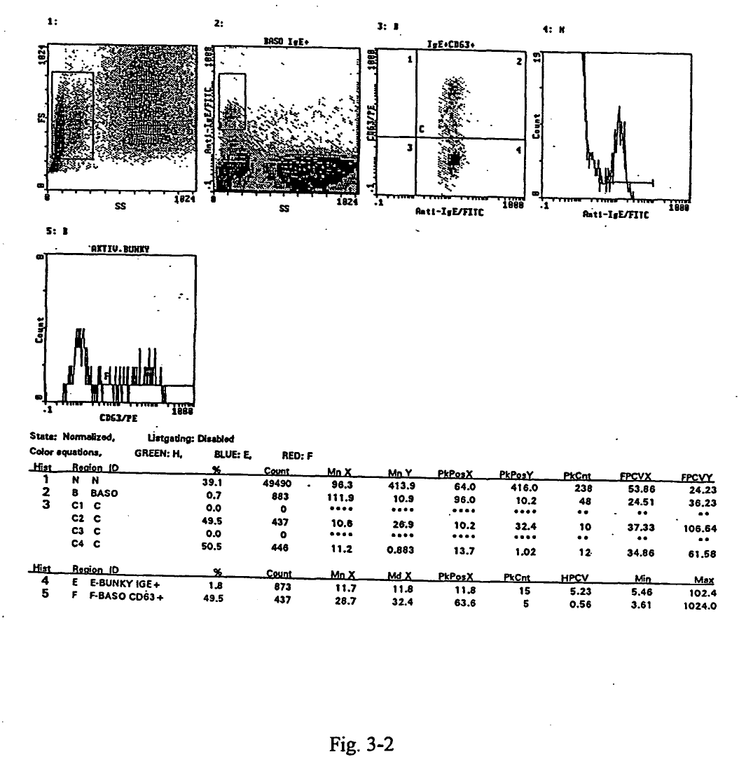

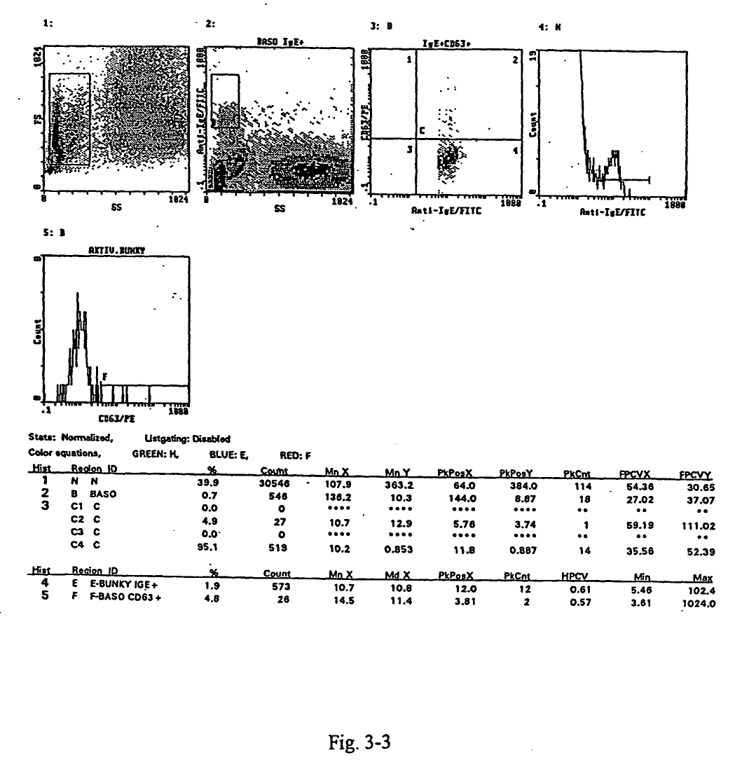

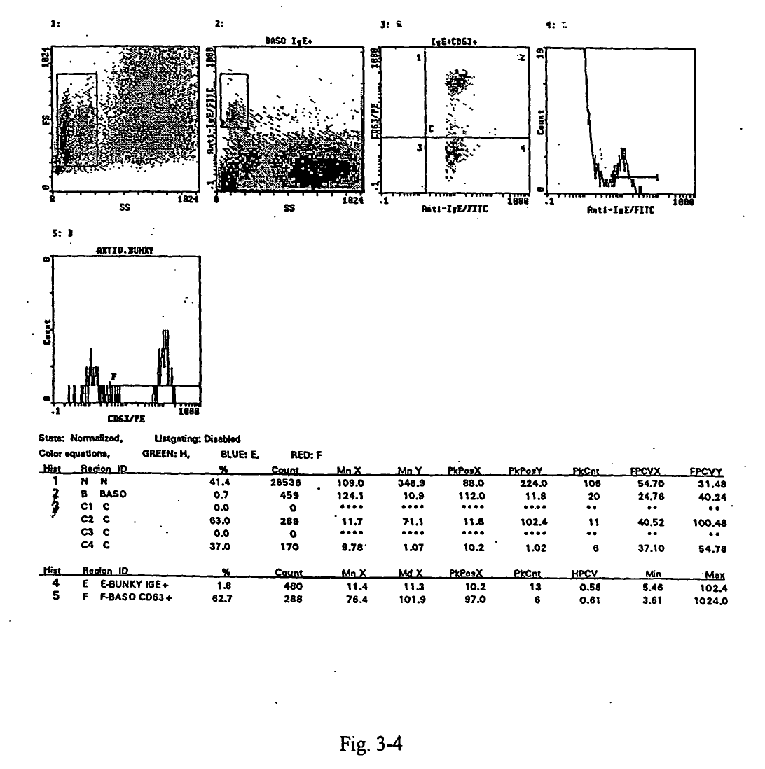

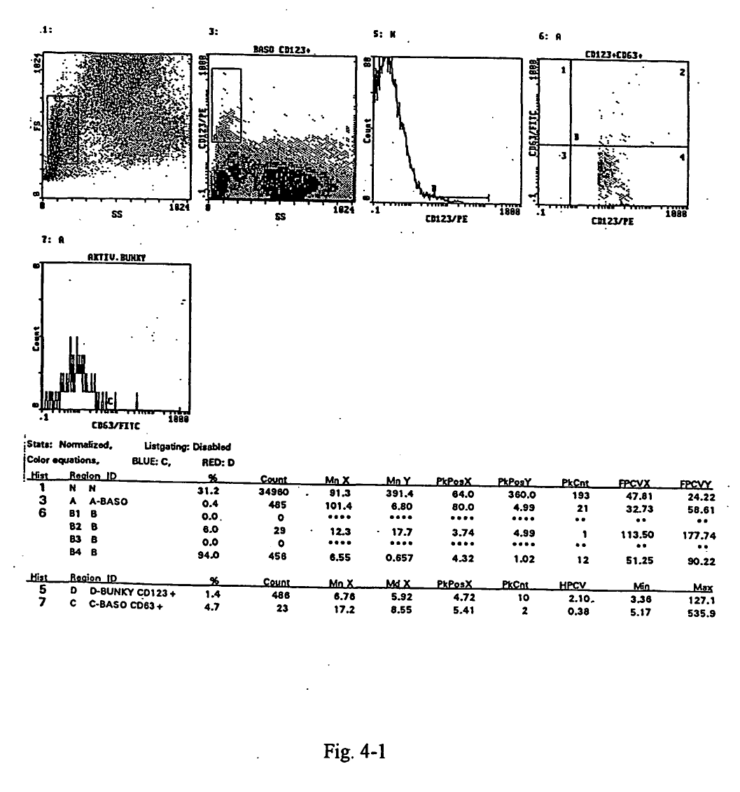

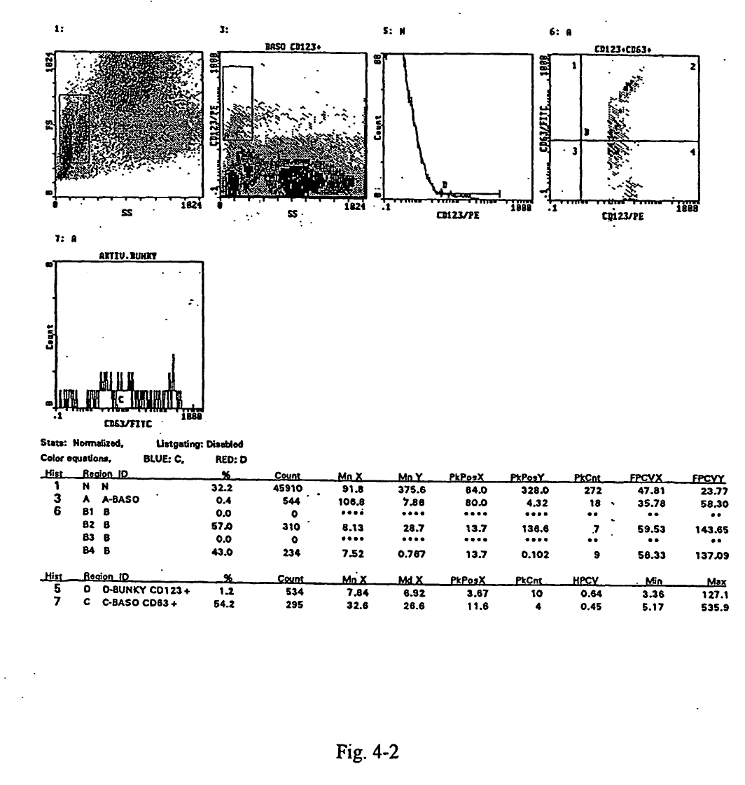

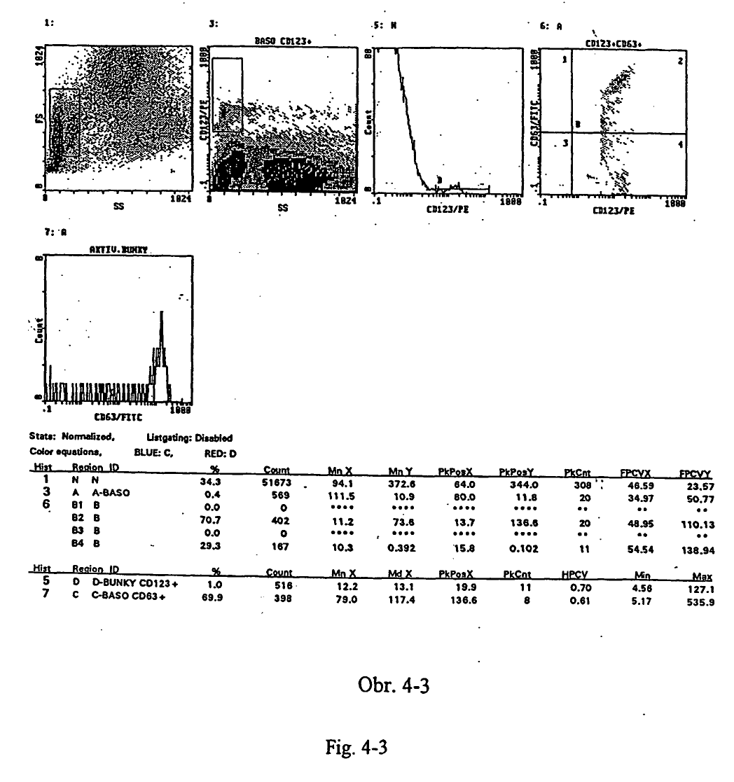

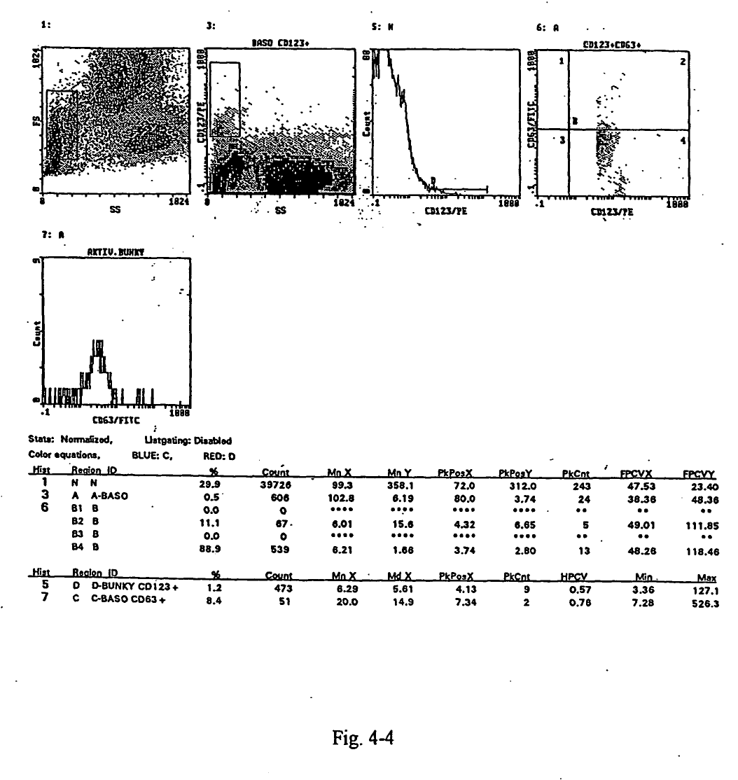

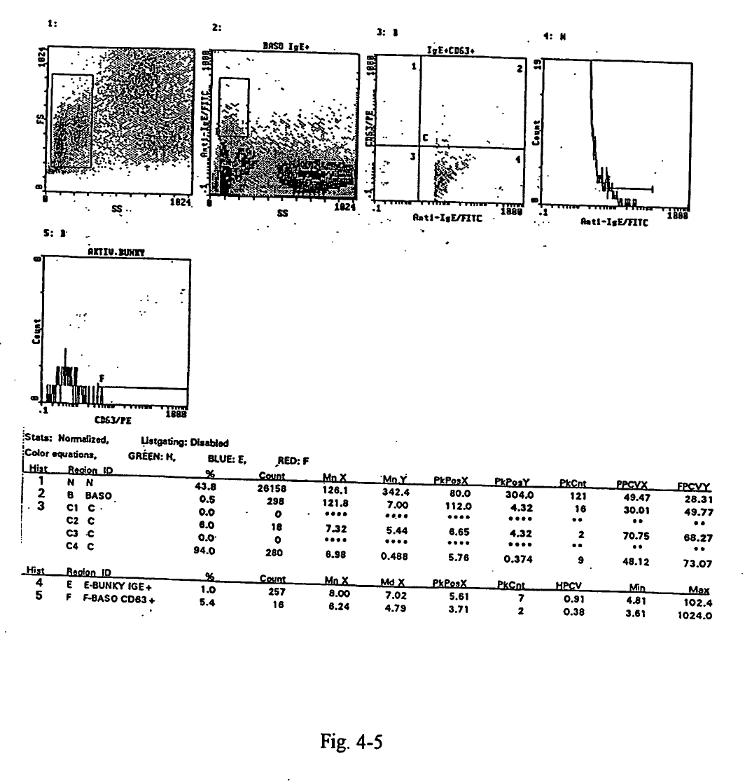

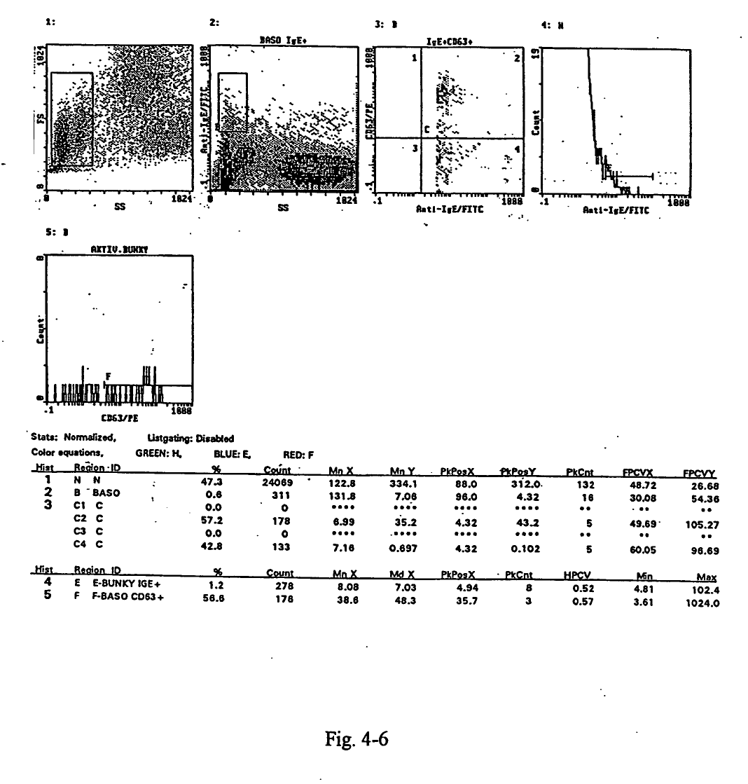

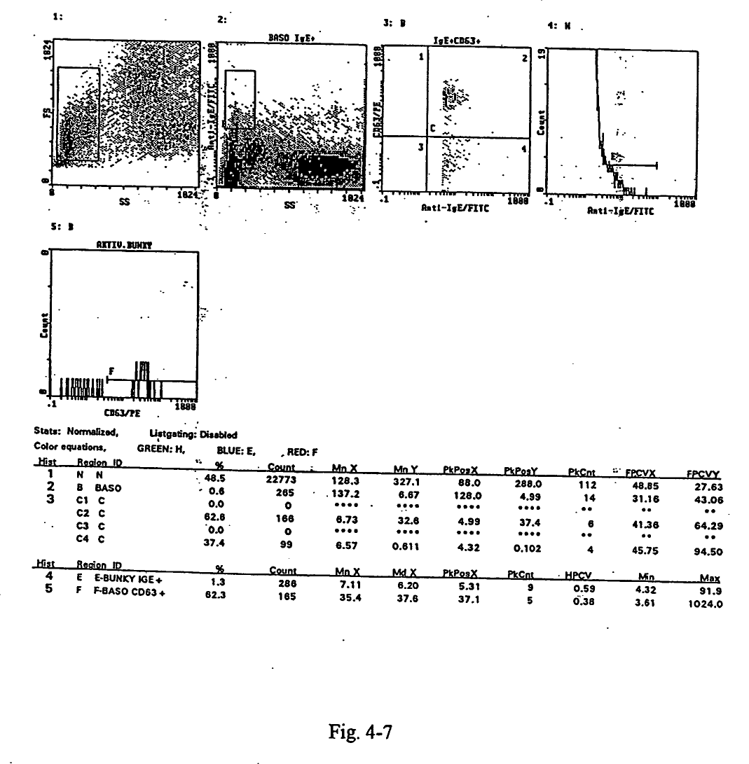

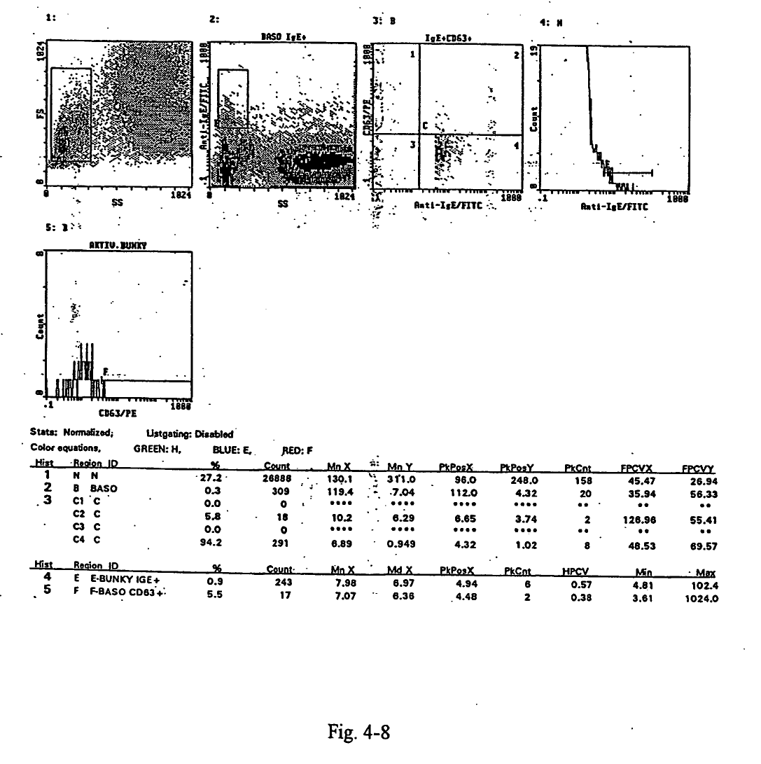

The meanings of the histograms in FIGS. 3-1 to 3-4:

Histogram 1—Distribution of the cells according to light scattering in the straight (FS) and lateral direction (SS). Gate N delineates the area wherein the basophils are present.

Histogram 2—Delineation of the basophil domain based on the light scattering in the side direction (SS) and on the binding of the anti-IgE/FITC antibody (gate B)

Histogram 3—Percentage distribution of the basophils according to the binding of the antibodies

Quadrant 1 (C1)—The cells stained with the CD63/PE antibody—other activated cells than basophils present in gate B (Histogram 2)

Quadrant 2 (C2)—The cells stained with both the anti-CD63/PE antibody and the anti-IgE/FITC antibody, i.e. the activated cells

Quadrant 3 (C3)—Unstained cells

Quadrant 4 (C4)—The cells stained with the anti-IgEIFITC antibody—non-activated basophils

Histogram 4—Distribution of the cells according to the fluorescence intensity, i.e. to the anti-IgE/FITC binding.

Histogram 5—Distribution of the cells by the fluorescence intensity, i.e. the CD63/PE binding.

FIGS. 4-1 to 4-8 illustrate the comparison of the results of anti-IgE/FITC-CD63/PE and anti-CD123/PE-CD63/FITC.

### EXAMPLES

### Example 1

The assay is carried out from whole blood collected into heparin. For each test 100 μl of the whole blood and 10 μl of an IL-3 solution in PBS (buffered physiological solution) at a concentration of 0.05 μg/ml is transferred by means of a pipette into a test tube. 100 μl PBS is added into the test tube for the negative control, 100 μl FMLP (chemotactic peptide N-formyl-L-methionyl-L-leucyl-L-phenylalanine) at a concentration of 0.433 μg/ml is added into the test tube for positive control, and 100 μl of the appropriately diluted allergen is added into the test tube with the sample to be tested. The samples are mixed thoroughly and incubated at 37 □C for 30 minutes. Then the samples are transferred into an ice bath. 20 μl of the monoclonal anti-CD123 antibody, labelled with PE (phycoerythrine; from Becton Dickinson), and 5 μl of the monoclonal antibody anti-CD63, labelled with FITC, from Caltag, are pipetted into each test-tube. The samples are mixed thoroughly and incubated in the ice bath for 15 to 20 minutes. Further processing is made at the room temperature. The erythrocytes in the samples are lysed by addition of a lysing agent, for example 2 ml NH₄Cl. After being lysed the samples are centrifuged at 1000 rpm. The samples are decanted and 0.5 ml PBS is added to the sediment. Measurement on the cytometer is made with the sample prepared in this manner.

### Example 2

The test is carried out from the whole blood collected into heparin For each test 100 μl of the blood and 10 μl of an IL-3 solution in PBS at a concentration of 0.05 μg/ml is pipette into a test tube. For the negative control 100 μl PBS, for the positive control 100 μl FMLP at a concentration of 0.433 μg/ml is added into the test tube. 100 μl of the appropriately diluted allergen is added into the test tube with the sample to be tested. The samples are mixed thoroughly and incubated at 37 □C for 30 minutes. Then the samples are transferred into an ice bath. 20 μl of the monoclonal antibody anti-CD203c labelled with PE from Immunotech, and 5 μl of the monoclonal antibody anti-CD63, labelled with FITC, from Caltag, are pipetted into each test-tube. The samples are mixed thoroughly and incubated in the ice bath for 15 to 20 minutes. Further processing is made at the room temperature.

The erythrocytes in the samples are lysed by addition of a lysing agent, for example 2 ml NH₄Cl. After being lysed the samples are centrifuged at 1000 rpm. The samples are decanted and 0.5 ml PBS is added to the sediment. Measurement on the cytometer is made with the sample prepared in this manner.

### Example 3

The test is carried out from the whole blood collected into heparin. For each test 100 μl of the blood and 10 μl of an IL-3 solution in PBS at a concentration of 0.5 μg/ml is pipetted into a test tube. For the negative control 100 μl PBS, for the positive control 100 μl FMLP at a concentration of 0.433 μg/ml is added into the test tube. 100 μl of the appropriately diluted allergen is added into the test tube with the sample to be tested. The samples are mixed thoroughly and incubated at 37 □C for 30 minutes. Then the samples are transferred into an ice bath. 20 μl of the monoclonal antibody anti-CD203c labelled with PE from Immunotech, and 5 μl of the monoclonal antibody anti-CD63, labelled with FITC, from Caltag, are pipetted into each test-tube. The samples are mixed thoroughly and incubated in the ice bath for 15 to 20 minutes. Further processing is made at the room temperature. The erythrocytes in the samples are lysed by addition of a lysing agent, for example 2 ml NH₄Cl. After being lysed the samples are centrifuged at 1000 rpm The samples are decanted and 0.5 ml PBS is added to the sediment. Measurement on the cytometer is made with the sample prepared in this manner.

### Example 4

The assay is made from a kit which contains 1 ml of a solution of IL-3 (Sigma) at a concentration of 0.05 μg/ml, 200 ml of PBS, 2 ml of the anti-CD63 antibody labelled with FITC (Beckman-Coulter), 2 ml of the anti-CD203c antibody labelled with PE (Beckman-Coulter), 10 ml of the solution of FMLP at a concentration of 4.33 μg/ml and 200 ml of a lysing solution NH₄Cl. The assay is carried out from the whole blood collected into heparin. For each test 100 μl of the whole blood and 10 μl of the IL-3 solution in PBS (buffered physiological solution) at a concentration of 0.05 μg/ml is pipetted into a test tube. 100 μl PBS is added into the test tube for the negative control, 100 μl FMLP at a concentration of 4.33 μg/ml is added into the test tube for the positive control, and 100 μl of the appropriately diluted allergen is added into the test tube with the sample to be tested. The samples are mixed thoroughly and incubated in at 37 □C for 30 minutes. Then the samples are transferred into an ice bath. 20 μl of the monoclonal antibody anti-CD203c, and 20 μl of the monoclonal antibody anti-CD63 are pipetted into each tube. The samples are mixed thoroughly and incubated in the ice bath for 15 to 20 minutes. Further processing is made at the room temperature. The erythrocytes in the samples are lysed by addition of a lysing agent, for example 2 ml NH₄Cl. After being lysed the samples are centrifuged at 1000 rpm The samples are decanted and 0.5 ml PBS is added to the sediment. Measurement on the cytometer is made with the sample prepared in this manner.

### Example 5

The assay is made from a kit which contains 1 ml of a solution of IL-3 (Sigma) at a concentration of 1 μg/ml, 200 ml of PBS, 2 ml of the anti-CD63 antibody labelled with FITC (Beckman-Coulter), 2 ml of the anti-CD203c antibody labelled with PE (Beckman-Coulter), 10 ml of the solution of FMLP at a concentration of 4.33 μg/ml and 200 ml of a lysing solution NH₄Cl. The assay is carried out from the whole blood collected into heparin. For each test 100 μl of the whole blood and 10 μl of the IL-3 solution in PBS (buffered physiological solution) at a concentration of 0.05 μg/ml is pipetted into a test tube. 100 μl PBS is added into the test tube for the negative control, 100 μl FMLP at a concentration of 4.33 μg/ml is added into the test tube for the positive control, and 100 μl of the appropriately diluted allergen is added into the test tube with the sample to be tested. The samples are mixed thoroughly aid incubated in at 37 □C for 30 minutes. Then the samples are transferred into an ice bath. 20 μl of the monoclonal antibody anti-CD203c, and 20 μl of the monoclonal antibody anti-CD63 are pipetted into each tube. The samples are mixed thoroughly and incubated in the ice bath for 15 to 20 minutes. Further processing is made at the room temperature. The erythrocytes in the samples are lysed by addition of a lysing agent, for example 2 ml NH₄Cl. After being lysed the samples are centrifuged at 1000 rpm The samples are decanted and 0.5 ml PBS is added to the sediment. Measurement on the cytometer is made with the sample prepared in this manner.

The results of the measurements on the flow cytometer Coulter Epics XL (Beckman-Coulter) are summarized in the following Tables and illustrated in attached Figures.

Key to abbreviations and terminology:

IL-3—interleukin-3,

PBS—buffered physiological solution,

FMLP—N-formyl-L-methionyl-L-leucyl-L-phenylalanine

FITC—fluorescein isothiocyanate

PE—phycoerythrin

DF—Dermathophagoides farinae (acarid)

DP—Dermathophagoides pteronyssinus (acarid)

gate, gating—in the field of cytometry: definition of the domain where e.g. the cells stained with monoclonal antibody are to be tested.

TABLE 1
Common expression of CD123+/IgE+ on the basophils as
function of the overall IgE concentration in the serum

| | | CD123+IgE+ | |
|---|---|---|---|
| Patient No. | IgE U/ml | gate IgE+ | gate CD123+ |
| MH | 48 | 79% | 56% |
| 18455 | 41.9 | 21% | 19% |
| 15931 | >17,800 | 32% | 80% |
| 17669 | 169 | 92% | 91% |
| 18394 | 428 | 38.6% | 84.5% |
| 18483 | 53 | 85% | 100% |
| 18539 | 251 | 72% | 87% |
| 18535 | 12.5 | 12% | 2.5% |
| 18507 | 11.1 | 6.2% | 3% |
| 18517 | >2,000 | 62.7% | 65.3% |
| 18765 | 66.5 | 81.4% | 73% |
| 12529 | 22 | 93% | 71% |
| 12580 | 395 | 88% | 80% |
| 12560 | 10.3 | small number of cells | 4.8% |
| 12530 | 95 | 68% | 69% |
| 19864 | 91 | 82.7% | 84.1% |
| JK | 200 | 91% | 85% |

The table and the attached FIGS. 1-1 to 1-8 give several examples of the two-color staining of the basophils. It was determined what percentage of the basophils stained with the anti-IgE antibody bear also the of CD123 receptor and, vice versa, what percentage of the basophils stained anti-CD123 are also anti-IgE positive.

It results from the table that staining of the basophils with the anti-IgE antibody is suitable for the serum levels of IgE 100-400 U/ml. At a low level of IgE (patients MH, 18535, 18455, 18507, 12560) the basophils stained with the anti-IgE cannot be gated, as the antibody does not bind to them. On the other hand, at a high level of IgE (patient No. 15931) either non-specific binding of the antibody to the monocytes occurs or the added antibody is displaced with the serum IgE.

TABLE 2
Common expression of CD203c+/IgE+ on the basophils
as function of the overall IgE concentration in the serum

CD203c+IgE+

| Patient No. | IgE U/ml | gate IgE+ | gate CD123c+ |
|---|---|---|---|
| 19894 | 91 | 77.4% | 82.3% |
| 19397 | 42.9 | 77.9% | 67.7% |
| 20156 | 42.5 | 92.7% | 90.7% |
| MH | 48 | 91.5% | 45.4% |
| JK | 200 | 87.1% | 97.3% |

The table and the attached FIGS. 2-1 to 2-5 give several examples of two-color staining of the basophils. It was determined what percentage of the basophils that are stained with the anti-ISE antibody bear also the of CD203c and, vice versa, what percentage of the basophils anti-CD203c stained are also anti-IgE positive.

Activation of the Basophils as a Function of the IL-3 Concentration

TABLE 3

| IL-3 | negative | non-specific | spec. stimulation |
|---|---|---|---|
| concentration | control | control | mixture of 3 grass |
| ng/ml | K0 | KN | species |
| 5 | 1.6% | 37.5% | 38.4% |
| 2.5 | 5.9% | 34.1% | 28.7% |
| 1.25 | 1.6% | 27.8% | 29.2% |
| 0.062 | 1.6% | 30.6% | 28.2% |
| 0 (PBS) | 1.0% | 15.2% | 3.9% |

TABLE 4

| IL-3 concentration | negative control | spec. stimulation |
|---|---|---|
| ng/ml | K0 | dog epithelium |
| 5 | 3.3% | 56.9% |
| 2.5 | 6.3% | 53.4% |
| 1.25 | — | 51.6% |
| 0.625 | — | 49.4% |
| 0.312 | 3.1% | 49.2% |
| 0.156 | — | 51.8% |
| 0.078 | 2.8% | 44.3% |
| 0.039 | — | 42.5% |
| 0 (PBS) | 6.4% | 40.7% |

TABLE 5

| IL-3 | negative | spec. | non-spec. |
|---|---|---|---|
| concentration | control | stimulation | stimulation |
| ng/ml | K0 | DF acarid | KN |
| 5 | 1.6% | 82.1% | 18.5% |
| 2.5 | 2.5% | 83.3% | 16.4% |
| 1.25 | 2.5% | 83.2% | 15.0% |
| 0.625 | 3.1% | 85.5% | 16.2% |
| 0.312 | 3.7% | 81.1% | 17.1% |
| 0.156 | 3.9% | 81.1% | 14.5% |
| 0 (PBS) | 2.6% | 17.0% | 10.9% |

TABLE 6

| conc. IL-3 | | |
|---|---|---|
| in the sample | Negative control | spec. stimulation |
| ng/ml | K0 | DF (acarid) |
| 41.3 | 2.8% | 81.9% |
| 20.6 | 4.3% | 85.7% |
| 10.3 | — | 87.7% |
| 5.16 | — | 82.8% |
| 2.58 | 5.1% | 78.5% |
| 1.29 | — | 67.2% |
| 0.65 | 3.8% | 51.3% |
| 0 (PBS) | 6.3% | 6.2% |

TABLE 7

| conc. IL-3 | | spec. stimulation |
|---|---|---|
| in the sample | Negative control | mixture of 3 grass |
| ng/ml | K0 | species |
| 41.3 | 3.4% | 86.6% |
| 20.6 | 5.3% | 86.7% |
| 10.3 | — | 86.0% |
| 5.16 | — | 80.9% |
| 2.58 | 5.1% | 78.6% |
| 1.29 | — | 66.1% |
| 0.65 | 4.5% | 62.0% |
| 0 (PBS) | 4.3% | 39.0% |

Activation of the Basophils as a Function of the Incubation Time with Allergen

TABLE 8

| | negative | spec. | non-spec. |
|---|---|---|---|
| Incubation time | control | stimulation | stimulation |
| in minutes | K0 | DF acarid | KN |
| 15 | 5.8% | 85.0% | 24.3% |
| 30 | 3.1% | 86.4% | 19.0% |
| 45 | 2.5% | 77.9% | 19.5% |

TABLE 9

| | | spec. stimulation | non-spec. |
|---|---|---|---|
| Incubation time | negative control | mixture of grass | stimulation |
| in minutes | K0 | species - ST | KN |
| 15 | 4.3% | 32.4% | 40.9% |
| 30 | 6.7% | 31.5% | 41.6% |
| 45 | 11.4% | 35.1% | 15.0% |

TABLE 10

| | negative | spec. | spec. | non-spec. |
|---|---|---|---|---|
| Incubation time | control | stimulation | stimulation | stimulation |
| in minutes | K0 | wasp | honey bee | KN |
| 15 | 6.6% | 88.1% | 8.4% | 23.4% |
| 30 | 4.7% | 89.1% | 11.9% | 37.3% |
| 45 | 6.7% | 78.5% | 10.5% | 28.5% |

TABLE 11

| | negative | spec. | spec. | non-spec. |
|---|---|---|---|---|
| Incubation time | control | stimulation | stimulation | stimulation |
| in minutes | K0 | DF acarid | egg white | KN |
| 15 | 2.4% | 7.6% | 6.8% | 41.6% |
| 30 | 2.9% | 4.5% | 7.4% | 42.1% |
| 45 | 4.8% | 13.0% | 4.6% | 22.4% |

Activation of the Basophils as a Function of Blood Pre-Incubation with IL-3

TABLE 12

| | negative | Spec. stimulation | Non-spec. |
|---|---|---|---|
| Incubation time | control | mixture of grass | stimulation |
| in minutes | K0 | species - ST | KN |
| 0 | 5.3% | 99.1% | 28.0% |
| 10 | 5.8% | 96.8% | 53.0% |

TABLE 13

| Pre- | | | | | Spec. | |
|---|---|---|---|---|---|---|
| incubation | negative | Spec. | Spec. | Spec. | stimulation | Non-specific |
| time - | control | stimulation | stimulation | stimulation | mixture of | stimulation |
| minutes | K0 | birch | celery | kiwi | grass species | KN |
| 0 | 2.7% | 94% | 94.7% | 11.9% | 98.4% | 48% |
| 10 | 4.1% | 94% | 94% | 12.5% | 99% | 74.5% |

TABLE 14

| | negative | Spec. stimulation | Non-spec. |
|---|---|---|---|
| Incubation time | control | mixture of grass | stimulation |
| in minutes | K0 | species - ST | KN |
| 0 | 5.3% | 99.1% | 28.0% |
| 10 | 5.8% | 96.8% | 53.0% |

Activation of the Basophils as a Function of the Allergen Dilution

TABLE 15

| | Allergen concentration 100 U/ml | | |
|---|---|---|---|
| Allergen | Dil. 5x | Dil. 10x | Dil. 20x |
| celery | 50.6% | 80.3% | 91% |
| birch | 46.5% | 46.2% | 46.3% |
| Wheat flour | 7.7% | 13.5% | 8.6% |

TABLE 16

| | Allergen concentration 100 U/ml | | | | |
|---|---|---|---|---|---|
| Allergen | Dil. 10x | Dil. 20x | Dil. 40x | Dil. 80x | Dil. 100x |
| wasp | 30.9% | 13.2% | 10.4% | — | — |
| honey bee | 29.7% | 40.8% | 50.0% | 41.5% | 36.8% |

TABLE 17

| | Allergen concentration 100 U/ml | | |
|---|---|---|---|
| Allergen | Dil. 10x | Dil. 100x | Dil. 1000x |
| apple | 52.8% | 7.5% | 1.0% |
| walnut | 72.6% | 57.2% | 5.6% |

TABLE 18

| | Allergen concentration 100 U/ml | | |
|---|---|---|---|
| Allergen | Dil. 10x | Dil. 100x | Dil. 1000x |
| Lolium | 31% | 25% | 21% |
| Timothy | 32% | 32.5% | 25% |

TABLE 19

| | Allergen concentration 100 U/ml | | |
|---|---|---|---|
| Allergen | Dil. 10x | Dil. 100x | Dil. 1000x |
| mixture of 3 grass | 42% | 43.5% | 54.3% |
| species | | | |

TABLE 20

| | Allergen concentration 100 U/ml | | |
|---|---|---|---|
| Allergen | Dil. 10x | Dil. 100x | Dil. 1000x |
| mixture of 3 grass | 39% | 10.7% | 6.7% |
| species | | | |

TABLE 21

| | Allergen concentration 100 U/ml | | |
|---|---|---|---|
| Allergen | Dil. 10x | Dil. 100x | Dil. 1000x |
| wasp | 54.8% | 58% | 11.2% |
| honey bee | 56.6% | 51.8% | 58.8% |

TABLE 22

| | Allergen concentration 100 U/ml | | |
|---|---|---|---|
| Allergen | Dil. 10x | Dil. 100x | Dil. 1000x |
| wasp | 59.8% | 67% | 15.4% |
| honey bee | 54.6% | 56.3% | 22.8 |

TABLE 23

Allergen concentration
100 U/ml

| Allergen | Dil 10x | Dil. 100x |
|---|---|---|
| wasp | 24.8% | 16.8% |
| honey bee | 6.2% | 11% |

Activation as a Function of IL-3 Concentration and Allergen Dilution

TABLE 24

| IL-3 | negative | | | Non-spec. |
|---|---|---|---|---|
| concentration | control | Spec. stim. | Spec. stim. | stimul. |
| ng/ml | K0 | h.bee 10x | h.bee 100x | KN |
| 2.5 | 6.9% | 85.2% | 94.7% | 41.1% |
| 1.25 | 3.4% | 77.1% | 75.2% | 39.4 |
| 0.625 | 2.2% | 86.9% | 80.2% | 42.4% |

Basic allergen concentration is 100 U/ml
Normal values: K0 <8%
with allergen <15%

Comparison of the Patient's Results Obtained by the Procedure Described (The Basophils Stained with Anti-IgE, Anti-CD123 or Anti-203) with the Test Named BASOTEST Available from ORPEGEN

Comparison: Anti-IgE/CD63 Versus Basotest

Pat1

| | negative | | | |
|---|---|---|---|---|
| | control | Spec. stim. | Spec. stim. | Spec. stim. |
| | K0 | birch | celery | mixture of grass |
| IgE/CD63 | 6.0% | 93% | 95.8% | 12.9% |
| Basotest | 5.5% | 87.8% | 90% | 13.5% |

Pat2

| | negative | Spec. | Spec. | Spec. | Spec. stim. |
|---|---|---|---|---|---|
| | control | stim. | stim. | stim. | mixture |
| | K0 | h.bee | wasp | birch | of grass |
| IgE/CD63 | 1.6% | 0.9% | 63.6% | 2.0% | 1.0% |
| Basotest | 3.6% | 1.0% | 68.1% | 1.6% | 1.3% |

Pat3

| | negative | | | |
|---|---|---|---|---|
| | control | Spec. stim. | Spec. stim. | Spec. stim. |
| | K0 | DF-acarid | DP-acarid | mixture of grass |
| CD123/CD63 | 6.4% | 11.8% | 11.7% | 12.3% |
| Basotest | 2.4% | 2.9% | 4.2% | 11.7% |

Pat4

| | negative | | | |
|---|---|---|---|---|
| | control | Spec. stim. | Spec. stim. | Non-spec. stim. |
| | K0 | wasp | h.bee | KN |
| CD123/CD63 | 2.3% | 8.5% | 5.2% | 46.1% |
| Basotest | 1.0% | 5.2% | 1.5% | 42.3% |

Comparison: Basotest Versus Anti-CD123/CD63, Anti-CD203c/CD63

Pat5

| | negative control | Non-spec. control | Spec. stim. |
|---|---|---|---|
| | K0 | KN | dog's epithelium |
| Basotest | 3.5% | 78.8% | 5.1% |
| CD123/CD63 | 5.8% | 68.6% | 10.7% |
| CD203/CD63 | 4.2% | 84.8% | 6.7% |

Comparison: Anti-IgE/CD63 Versus Basotest, Anti-CD123/CD63, AntiCD203c/CD63

Pat6

| | | negative | Non-spec. | |
|---|---|---|---|---|
| | CD123 or | control | control | Spec. stim. |
| | CD203/IgE | K0 | KN | DF-acarid |
| IgE/CD63 | — | 3.1% | 19.0% | 86.4% |
| Basotest | — | 5.4% | 19.0% | 85.9% |
| CD123/CD63 | 85% | 2.4% | 32.0% | 85.3% |
| CD203/CD63 | 96.7% | 5.9% | 36.5% | 92.8% |

Comparison: Anti-IgE/FITC-CD63/PE Anti-CD123/PE-CD63/FITC, and CD203c/PE-CD63/FITC.

Hypersensitivity to the h.bee and wasp allergens was tested. The results are illustrated in FIGS. 3-1 to 3-4 and expressed as percentages of the positive basophils, i.e. of those which are sensitive to an allergen (in the figures, histogram 3 or 6, quadrant 2—cells which bear both the marker of the basophil and the CD63 activation antigen). K0—sample without stimulation (negative control)

| cell labelling | K0 % | wasp % | h.bee % |
|---|---|---|---|
| IgE/CD63 | 5.1 | 5.0 | 61.8 |
| CD123/CD63 | 1.5 | 3.4 | 56.6 |
| CD203c/CD63 | 3.5 | 6.2 | 50.3 |

Normal values: K0 < 8%
with allergen <15%

For comparison, specific IgE antibodies against the following allergens have been determined for the patient:

| | | |
|---|---|---|
| wasp | 0.35 U/ml | |
| h.bee | 1.4 U/ml | positive are the values >0.35 U/ml |

Due to the level of the overall IgE (about 90 IU/ml) all the three modes of staining of the basophils are appropriate, with comparable results.

Staining of the cells with antibodies against IgE (anti-IgE/FITC) and against the activation antigen CD63 (CD63/PE).

Comparison of Results: Anti-IgE/FITC-CD63/PE Versus Anti-CD123/PE-CD63/FITC.

Hypersensitivity to the allergens of egg white, cow's milk, and wheat flour has been tested.

The results are illustrated in the FIGS. 4-1 to 4-8 and expressed as percentages of the positive basophils, i.e. of those which are sensitive to the allergen (in the figures, histogram 3 or 6, quadrant 2—cells which bear both the marker of the basophil and the activation antigen CD63), K0—sample without stimulation (negative control).

| Basophil labelling | K0 % | egg white % | cow's milk % | wheat flour % |
|---|---|---|---|---|
| IgE/CD63 | 6.0 | 57.2 | 62.6 | 5.8 |
| CD123/CD63 | 6.0 | 57 | 70.7 | 11.1 |

Normal values: K0 < 8%
with allergen <15%

For comparison, specific IgE antibodies against the following allergens have been determined for the patient:

| | |
|---|---|
| egg white | >25 U/ml |
| cow's milk | >25 U/ml |
| wheat flour | 1.71 U/ml; |
| | positive are values >35 U/ml |

Because of the high level of the total IgE (>17,800 IU/ml) it is more suitable to stain the basophils with anti-CD123 than with an anti-IgE antibody (its binding to serum. IgE apparently occurs).

The discrepance between the results of the spec. IgE against wheat flour (1.7-possitive) and the non-hypersensitivity found by us confirms our finding that during determination of sIgEs the non-specific binding occurs. The cross-reacting antibodies are simultaneously determined (false positive results), which antibodies have lower affinities and do not often cause clinical reactions.

Comparison of the Results of Activation of the Basophils, sIgE and Clinical Manifestations

Allergen: h.bee

| | % of the basophils | Specific IgE against | Clinical |
|---|---|---|---|
| Patient | activated | honey bee | manifestations |
| Ce-I | 51.8% | 14.6 | SSR |
| Ce-D | 56.3% | 1.4 | SSR |
| Bo | 20% | <0.35 | SLR |
| Ho | 5.2% | <0.35 | ? |
| En | 6.2% | <0.35 | ? |
| Ben | 82% | 1.33 | SSR |
| Hav | 5.7% | <0.35 | ? |
| Sev | 5.6% | <0.35 | ? |
| Sch | 77% | >17.5 | ? |
| Fru | 68.5% | >17.5 | ? |
| Bo | 40.8% | >17.5 | ? |
| Vla | 1.8% | <0.35 | ? |
| Vl | 1.7% | <0.35 | ? |
| Ja | 5.9% | 0.35 | ? |
| Kr | 2.5% | 0.75 | ? |
| Pru | 78.9% | 0.52 | ? |
| Tu | 2.0% | 0.65 | ? |
| He | 8.2% | <0.35 | ? |
| Ha | 11.9% | <0.35 | ? |
| Ba | 57.4% | 1.4 | SSR |
| Sko | 10.3% | 0.36 | ? |

SSR—severe system reaction,
SLR—severe local reaction
Positive results:
>0.35 U/ml sIgE
>15% activated basophils

Allergen: Wasp

| | % of the basophils | Specific IgE against | Clinical |
|---|---|---|---|
| Patient | activated | wasps | manifestations |
| Va | 88% | >17.5 | SSR |
| Ce-I | 58% | <0.35 | SSR |
| Ce-D | 59.9 | 5.9 | SSR |
| Bo | 30% | <0.35 | SLR |
| Ho | 7% | <0.35 | ? |
| En | 24.8% | 0.51 | ? |
| Ben | 8.5% | <0.35 | ? |
| Hav | 57.8% | 7.33 | SSR |
| Sev | 58% | <0.35 | SSR |
| Sch | 86% | >17.5 | SSR |
| Fru | 74.3% | 1.04 | ? |
| Bo | 30.9% | 1.33 | ? |
| Vla | 3.2% | <0.35 | ? |
| Vl | 2.1% | <0.35 | ? |
| Ja | 27.6% | 0.59 | ? |
| Kr | 71.5% | 0.75 | SSR |
| Pru | 78% | 0.35 | ? |
| Tu | 7.1% | 0.42 | ? |
| He | 76.1% | <0.35 | SSR |
| Ha | 89% | 0.87 | SSR |
| Ba | 8.1% | <0.35 | ? |
| Sko | 49.7% | <0.35 | SSL |

SSR—severe system reaction,
SLR—severe local reaction
Positive results:
>0.35 U/ml sIgE
>15% activated basophils

1-6. Cancelled.

7. A method for determining hypersensitivity of an individual to an allergen, the method comprising:
a) incubating a mixture comprising a blood sample from an individual, interleukin-3, an allergen and a physiological buffer at a physiological temperature for the individual, and
b) measuring the level of CD63 expression on basophils in the incubated mixture of a), wherein the basophils are identified by expression of CD123 or CD203c, wherein CD63 expression on basophils indicates basophil activation and hypersensitivity of the individual to the allergen.

8. The method of claim 7, wherein the CD63 expression is measured using an anti-CD63 antibody.

9. The method of claim 7, wherein the CD123 or CD203c expression is determined using an anti-CD123 antibody or anti-CD203c antibody.

10. The method of claim 7, wherein the CD63 expression is measured using a labeled anti-CD63 antibody, wherein the CD123 expression is determined using a labeled anti-CD123 antibody, and wherein the anti-CD63 antibody and the anti-CD123 antibody have labels that are distinguishable from each other.

11. The method of claim 7, wherein the CD63 expression is measured using a labeled anti-CD63 antibody, wherein the CD203c expression is determined using a labeled anti-CD203c antibody, and wherein the anti-CD63 antibody and the anti-CD203c antibody have labels that are distinguishable from each other.

12. The method of claim 8, wherein the anti-CD63 antibody is a monoclonal antibody.

13. The method of claim 9, wherein the anti-CD123 antibody or anti-CD203c antibody is a monoclonal antibody.

14. The method of claim 7, wherein the level of CD63 expression on basophils is measured using flow cytometry.

15. The method of claim 14, wherein the basophils are identified using flow cytometry.

16. The method of claim 7, wherein the basophils are identified using flow cytometry.

17. A kit for use in determining hypersensitivity through measure of basophil activation, the kit comprising:
interleukin-3, a labeled anti-CD63 antibody, a labeled anti-basophil marker antibody selected from an anti-CD123 antibody or an anti-CD203c antibody, and a non-specific basophil activator,
wherein the labeled anti-CD63 antibody and the labeled anti-basophil marker antibody have labels that are distinguishable from each other.

18. The kit according to claim 17, wherein the non-specific basophil activator is N-formyl-L-methionyl-L-leucyl-L-phenylalanine.

19. The kit according to claim 17, further comprising at least one an allergen.

20. The kit according to claim 19, wherein the allergen is an insect allergen.

21. The kit according to claim 20, wherein the insect allergen is a honey bee allergen or a wasp allergen.

22. The kit according to claim 19, wherein the allergen is a food allergen.

23. The kit according to claim 22, wherein the food allergen is selected from the group consisting of egg white allergen, cow's milk allergen, wheat flour allergen, celery allergen, walnut allergen, apple allergen and kiwi allergen.

24. The kit according to claim 19, wherein the allergen is a tree allergen.

25. The kit according to claim 24, wherein the tree allergen is a birch allergen.

26. The kit according to claim 19, wherein the allergen is a grass allergen.
